(19) United States
(12) Plant Patent Application Publication
(10) Pub. No.: US 2004/0010829 P1
(43) Pub. Date: Jan. 15, 2004

(54) MINT PLANT NAMED 'MCKENZIE'

(76) Inventor: Mark Morris, Eugene, OR (US)

Correspondence Address: KRAMER & AMADO, P.C., 2001 JEFFERSON DAVIS HWY, SUITE 1101, ARLINGTON, VA, 22202, US

(21) Appl. No.: 10/193,191

(22) Filed: Jul. 12, 2002

(51) Int. Cl. A01H 5/00

(52) U.S. Cl. PLT/259

(57) ABSTRACT

A new and distinct peppermint plant ‘MCKENZIE’ characterized by its upright growth habit, vigorous and abundant root growth, resistance to Spider Mites (Tetranychus urticae), Mint rust (Puccinia menthe Pers), Mint wilt (Verticillium dahliae and Verticillium albo-atrum), and a distinct flower spike with mostly white petals and consistent oil quality throughout its phases of maturity.

### FIELD OF THE INVENTION

The present invention relates to a new and distinct peppermint plant botanically known as Mentha×piperita. The new variety has been named ‘MCKENZIE’ and will be referred as such hereafter.

### BACKGROUND OF THE INVENTION

The new peppermint plant of the present invention was developed by the A.M. Todd Company variety development team in an effort to cultivate a peppermint variety exhibiting an upright growth habit, vigorous and abundant root growth, greater resistance to Spider mites (Tetranychus urticae), Mint rust (Puccinia menthe Pers) and Verticillium wilt (Verticillium dahliae and V. albo-atrum). Further, MCKENZIE possesses peppermint oil features that are only minimally changed in oil profile based on plant maturity.

The inventor's explored ways of creating peppermint hybrids through conventional crosses without the use of chemicals to increase ploidy. The method relied on adjusting environmental conditions to generate viable pollen. Using this method, several seedlings were generated in 1996 using two peppermint plants as parents.

The resulting selection has been under continuous evaluation at the A.M. Todd facility located in Jefferson, Oreg. since 1996 and has been asexually propagated through stem and rhizome cuttings. The resulting propagules have remained identical to the parent plant in appearance, resistance to the common diseases encountered by mint plants, oil quality and vigorous root growth.

### SUMMARY OF THE INVENTION

The present invention relates to a novel peppermint plant named ‘MCKENZIE’ characterized by its more upright growth habit as shown by taller growth with thicker stems, when compared to commercial peppermint varieties, more abundant and vigorous root growth and resistance to Mint rust (Puccinia menthe Pers), Verticillium wilt (Verticillium dahliae and V. albo-atrum), and Spider mites (Tetranychus urticae). The peppermint oil profile of MCKENZIE is also similar throughout its maturity.

### BRIEF DESCRIPTION OF THE PHOTOGRAPHS

The accompanying color photographs of ‘MCKENZIE’ show the new variety:

FIG. 1: shows the new peppermint plant ‘MCKENZIE’

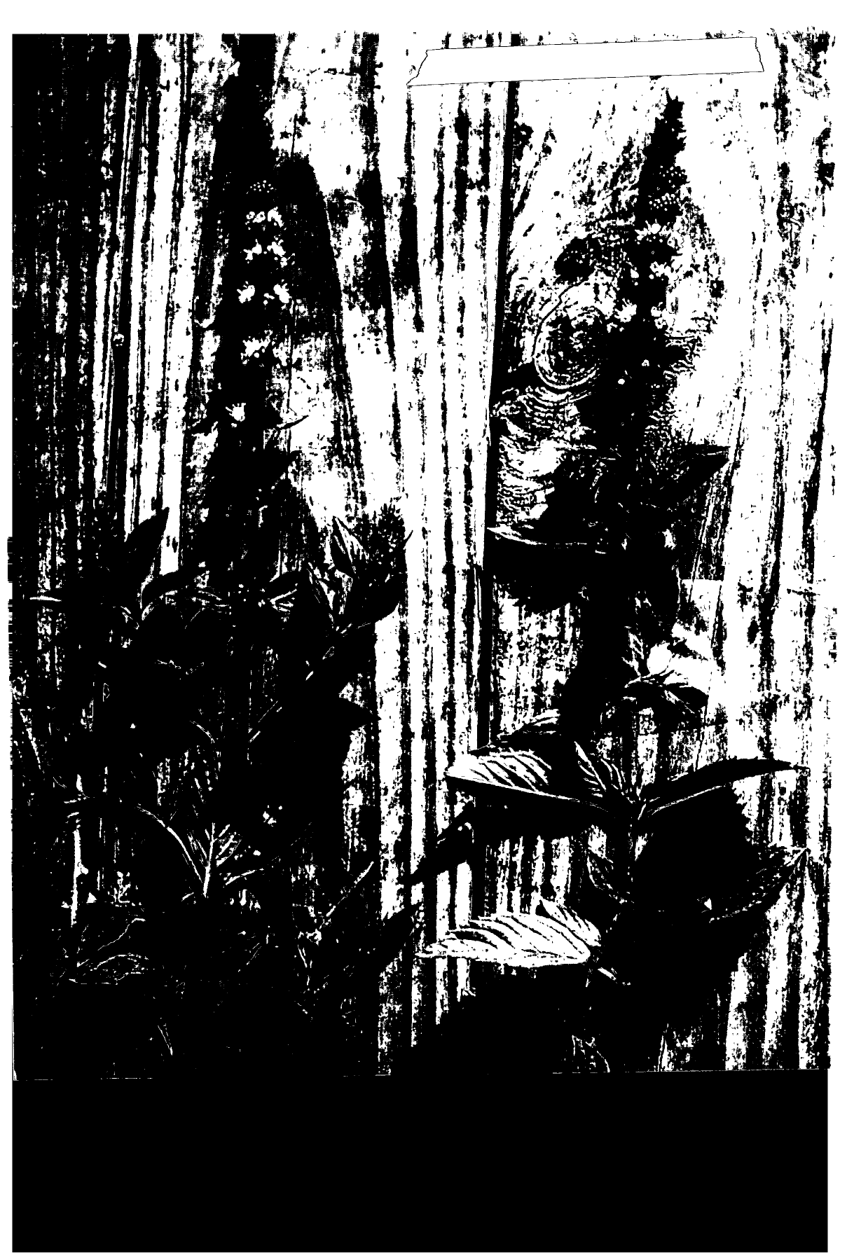

FIG. 2: shows the flower spike of ‘MCKENZIE’ and its flower whorls having mostly white flower petals.

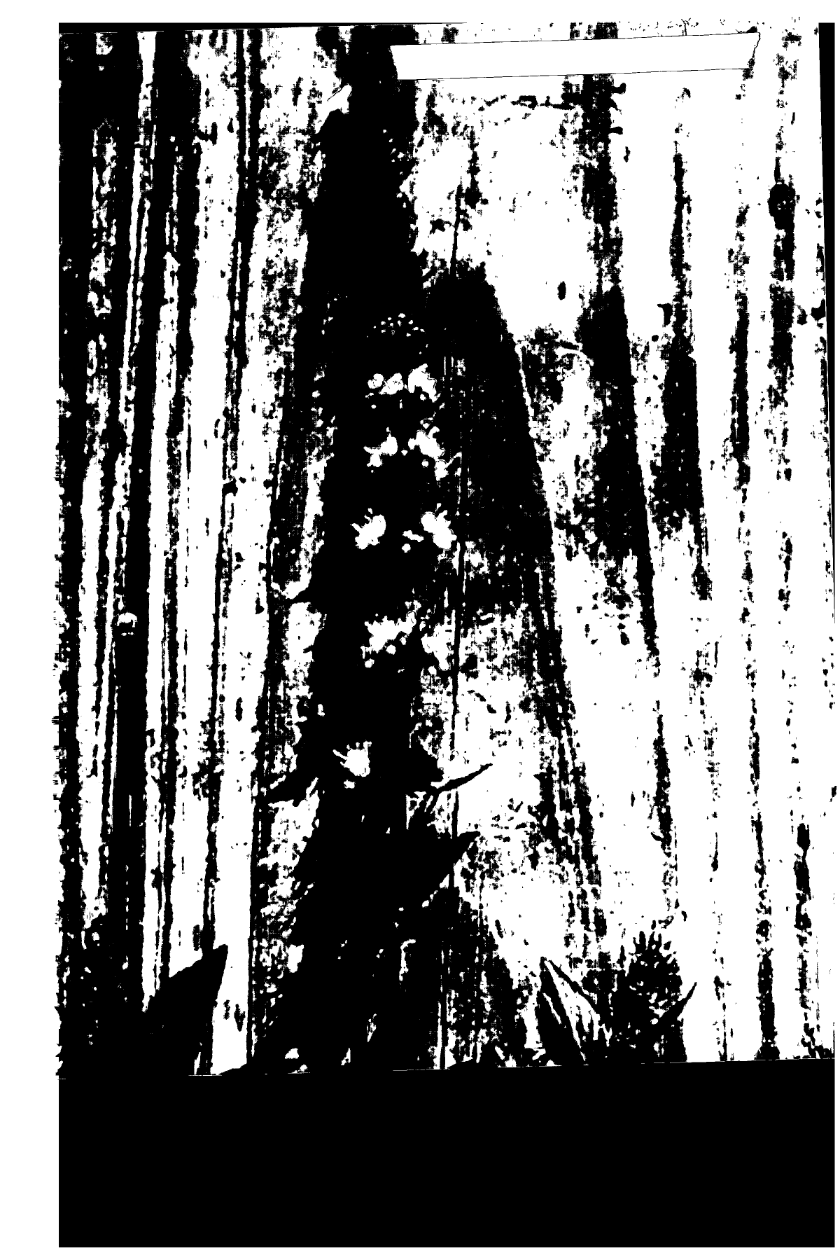

FIG. 3: shows the mature leaves of ‘MCKENZIE’.

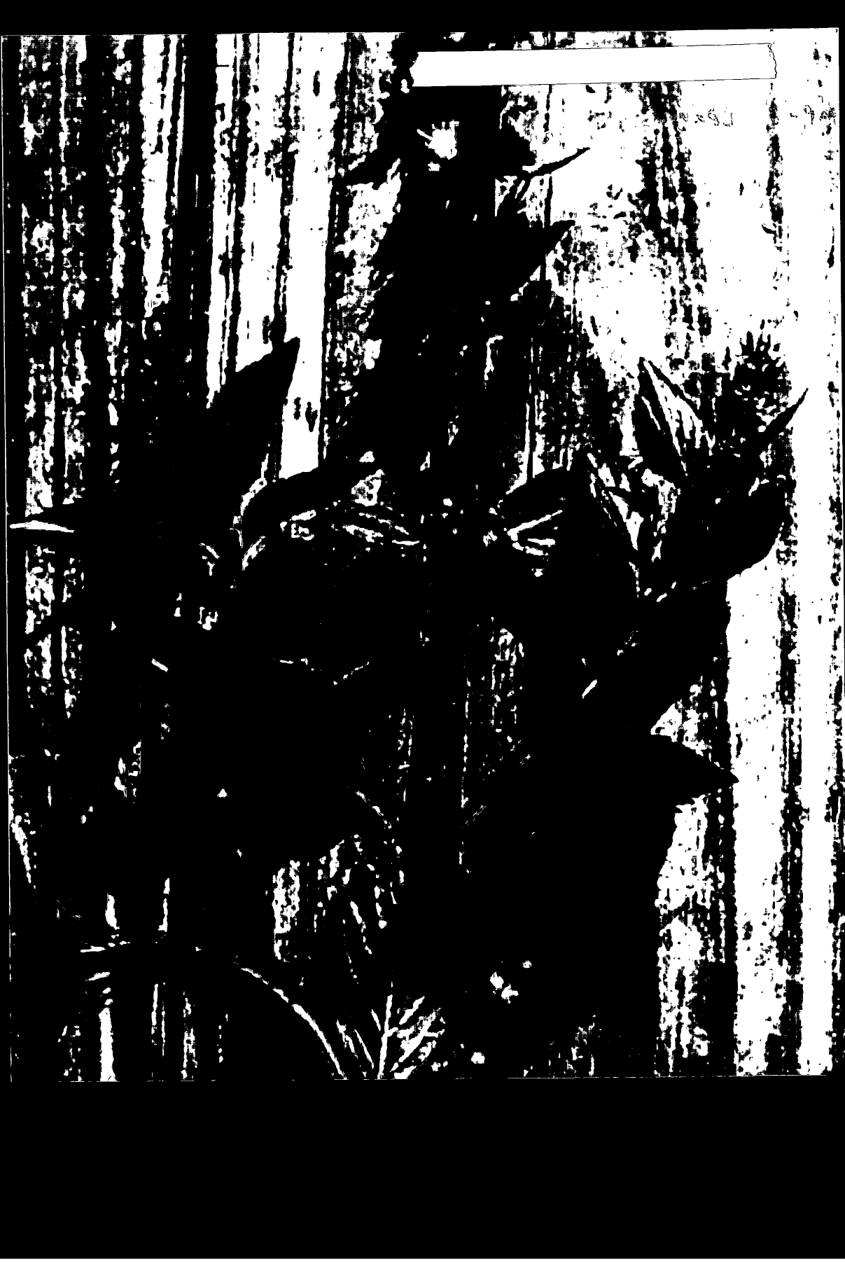

### DETAILED DESCRIPTION OF THE INVENTION

The present invention is a new and distinct variety of peppermint plant having the following characteristics that, in combination, are not exhibited in other peppermint plants:

1. More upright growth habit;

2. More vigorous and abundant root growth;

3. Improved resistance to Spider mites (Tetranychus urticae);

4. Oil profile that shows little variability based on plant maturity.

5. Improved resistance to Mint rust (Puccinia menthe Pers);

6. Improved resistance to Verticillium wilt (Verticillium dahliae and V. albo-atrum).

MCKENZIE is an herbaceous perennial with an upright shrubby growth habit. This facilitates harvesting and thereby reduces costs. Numerous branching stems are produced each year with an eventual height between 70-90 cm. When compared to Black Mitcham, the industry standard, MCKENZIE is more upright in its growth habit, taller with thicker stems (due to a large extent to the highly lignified stems which reduces lodging), larger leaves and a lighter green color with less anthocyanin pigment development under similar growing conditions. Based on flowering MCKENZIE matures at approximately the same time as Black Mitcham. MCKENZIE also features the additional benefit of vigorous and abundant root growth, which results in more rapid acreage expansion and reduction in planting costs.

In botanical terms, MCKENZIE has a distinct flower spike that is quite elongated as compared to other peppermints and can be up to 30 cm long under some growing conditions when measured from the lowermost verticillaster to the tip. The number of paired flower whorls, or verticillasters, can vary from 8 to 20. The first internode between verticiels varies from 1.8 to 4.0 cm. Flower petal color is much lighter than Black Mitcham. The petals are mostly white, but have a very slight lavender cast to them. The leaves halfway up the main stem are approximately 7.5 cm long by 2.4 cm wide and have an ovate shape with a serrate margin.

The oil produced by MCKENZIE is of a typical peppermint type. In contrast to Black Mitcham, the oil profile of the MCKENZIE varies little with plant maturity. This feature allows for increased yield due to flexible harvest timing during a single season without significant changes occurring to its oil profile.

MCKENZIE has been grown and observed under 3 different field conditions as well as under greenhouse conditions. As with all plants, the traits have varied depending on location. These differences can be attributed to varying climates, soils, fertilizers and water regimes. However, MCKENZIE remains distinct from other peppermint plants in its characteristics. The resistance to the diseases identified above is presented in Table I. The results were gathered from a study performed in western Oregon and show the comparison of MCKENZIE to Black Mitcham according to the mean number of rust pustules per leaf, mean number of Verticillium strikes per plot and mean number of spider mites per leaf during three years of analysis.

TABLE I

| | Mean Number of | Mean Number | Mean Number of |
|---|---|---|---|
| | Rust Pustules | of Verticillium | Spider Mites per |
| Variety | per leaf | strikes per plot | leaf |
| Aug. 15, 1999 | | | |
| MCKENZIE | 0.00 | 0.00 | 0.75 |
| Black Mitcham | 40.30 | 9.25 | 8.25 |
| Aug. 22, 2000 | | | |
| MCKENZIE | 0.00 | 0.75 | 0.58 |
| Black Mitcham | 183.25 | 17.00 | 10.00 |
| Aug. 26, 2001 | | | |
| MCKENZIE | 0.25 | 1.75 | 0.25 |
| Black Mitcham | 81.25 | 30.50 | 11.25 |

As illustrated by Table I, MCKENZIE is significantly more resistant to the diseases shown. For example, although the incidence of Mint Rust varied overall according to the three years shown due to differences in annual growing conditions, MCKENZIE consistently showed much greater resistance to Puccinia menthe Pers when compared to the mean number of Rust Pustules per leaf. In fact, Mint Rust was almost eliminated. Similarly, the number of mean strikes per plot for Verticillium dahliae and Verticillium albo-atrum was markedly reduced by MCKENZIE when compared to Black Mitcham. In the case of Tetranychus urticae, the man number of Spider Mites per leaf was, again, almost eliminated in comparison to Black Mitcham.

While the plant identified in the present invention has been described as it relates to a specific embodiment, it is understood that this application is intended to cover other variations, uses and adaptations that may arise under different environmental conditions.

What is claimed is:

1. A new and distinct variety of peppermint plant, substantially as shown and described.
